(19) United States
(12) Patent Application Publication
(10) Pub. No.: US 2025/0185995 A1
(43) Pub. Date: Jun. 12, 2025

(54) AUTONOMOUS DRIVING BASED ON HEALTH OF AN OCCUPANT OF A VEHICLE

(71) Applicant: Volvo Car Corporation, Göteborg (SE)

(72) Inventors: Kristina Ek Blohm, Torslanda (SE); Matthias Le Saux, Göteborg (SE); Jonas Carlsson, Mölndal (SE); Mikael Törmänen, Gothenburg (SE)

(21) Appl. No.: 18/536,505

(22) Filed: Dec. 12, 2023

(51) Int. Cl. A61B 5/00; A61B 5/0205; A61B 5/145; A61B 5/1455; A61B 5/332; B60W 50/14; B60W 60/00

(52) U.S. Cl. CPC A61B 5/6893; A61B 5/0205; A61B 5/746; A61B 5/747; B60W 50/14; B60W 60/0051; A61B 5/0077; A61B 5/14532; A61B 5/14551; A61B 5/332; B60W 2420/403; B60W 2540/221; B60W 2556/45

(57) ABSTRACT

Autonomous driving based on health of an occupant of a vehicle (e.g., using a computerized tool) is enabled. For example, a method can comprise, using a health sensor, determining, by a vehicle comprising a processor, one or more health metrics associated with an occupant of the vehicle, based on a defined activation criterion associated with the one or more health metrics being determined to be satisfied, activating, by the vehicle, an autonomous driving mode, in response to activation of the autonomous driving mode, determining, by the vehicle, a route from a current location of the vehicle to a defined location, wherein the defined location is determined based on the one or more health metrics, and using the route, autonomously navigating, by the vehicle, the vehicle to the defined location.

### TECHNICAL FIELD

The disclosed subject matter relates to autonomously driven vehicles (e.g., transportation vehicles) and, more particularly, to autonomous driving based on health of an occupant of a vehicle.

### BACKGROUND

The dangers of a driver of a vehicle becoming incapacitated are profound and potentially life-threatening. When a driver (or another occupant of a vehicle) loses consciousness or control of a vehicle, for instance, due to a medical emergency, fatigue, or impairment, they pose a significant risk to themselves and others on the road. Incapacitated drivers can cause accidents, leading to injuries or fatalities, as they are unable to make split-second decisions or react to changing traffic conditions.

The above-described background relating to vehicular safety is merely intended to provide a contextual overview of some current issues and is not intended to be exhaustive. Other contextual information may become further apparent upon review of the following detailed description.

### SUMMARY

The following presents a summary to provide a basic understanding of one or more embodiments of the invention. This summary is not intended to identify key or critical elements, or delineate any scope of the particular embodiments or any scope of the claims. Its sole purpose is to present concepts in a simplified form as a prelude to the more detailed description that is presented later. In one or more embodiments described herein, systems, devices, computer-implemented methods, apparatuses and/or computer program products that facilitate vehicle battery health optimization are described.

As alluded to above, vehicular safety can be improved in various ways, and various embodiments are described herein to this end and/or other ends.

According to an embodiment, a vehicle can comprise a memory that stores computer executable components, and a processor that executes the computer executable components stored in the memory, wherein the computer executable components can comprise a health component that, using a health sensor, determines one or more health metrics associated with an occupant of the vehicle, an activation component that, based on a defined activation criterion associated with the one or more health metrics being determined to be satisfied, activates an autonomous driving mode, a navigation component that, in response to activation of the autonomous driving mode, determines a route from a current location of the vehicle to a defined location, wherein the defined location is determined based on the one or more health metrics, and an autonomous driving component that, using the route, autonomously navigates the vehicle to the defined location.

According to another embodiment, a method can comprising, using a health sensor, determining, by a vehicle comprising a processor, one or more health metrics associated with an occupant of the vehicle, based on a defined activation criterion associated with the one or more health metrics being determined to be satisfied, activating, by the vehicle, an autonomous driving mode, in response to activation of the autonomous driving mode, determining, by the vehicle, a route from a current location of the vehicle to a defined location, wherein the defined location is determined based on the one or more health metrics, and using the route, autonomously navigating, by the vehicle, the vehicle to the defined location.

According to yet another embodiment, a non-transitory machine-readable medium can comprise executable instructions that, when executed by a processor, facilitate performance of operations, comprising, using a health sensor, determining one or more health metrics associated with an occupant of a vehicle, based on a defined activation criterion associated with the one or more health metrics being determined to be satisfied, activating an autonomous driving mode of the vehicle, in response to activation of the autonomous driving mode, determining a route from a current location of the vehicle to a defined location, wherein the defined location is determined based on the one or more health metrics, and using the route, autonomously navigating the vehicle to the defined location.

### BRIEF DESCRIPTION OF DRAWINGS

FIG. 1 illustrates a block diagram of an exemplary system in accordance with one or more embodiments described herein.

FIG. 2 illustrates a block diagram of example, non-limiting computer executable components in accordance with one or more embodiments described herein.

FIG. 3 illustrates a block diagram of example, non-limiting vehicle electronic systems/devices in accordance with one or more embodiments described herein.

FIG. 4 illustrates a diagram of an example, non-limiting vehicle interior in accordance with one or more embodiments described herein.

FIG. 5 illustrates an exemplary scenario in accordance with one or more embodiments described herein.

FIG. 6 illustrates an exemplary scenario in accordance with one or more embodiments described herein.

FIG. 7 illustrates a block flow diagram for a process associated with autonomous driving based on health of an occupant of a vehicle in accordance with one or more embodiments described herein.

FIG. 8 is an example, non-limiting computing environment in which one or more embodiments described herein can be implemented.

FIG. 9 is an example, non-limiting networking environment in which one or more embodiments described herein can be implemented.

### DETAILED DESCRIPTION

The following detailed description is merely illustrative and is not intended to limit embodiments and/or application or uses of embodiments. Furthermore, there is no intention to be bound by any expressed or implied information presented in the preceding Background or Summary sections, or in the Detailed Description section.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

It will be understood that when an element is referred to as being “coupled” to another element, it can describe one or more different types of coupling including, but not limited to, chemical coupling, communicative coupling, capacitive coupling, electrical coupling, electromagnetic coupling, inductive coupling, operative coupling, conductive coupling, acoustic coupling, ultrasound coupling, optical coupling, physical coupling, thermal coupling, and/or another type of coupling. As referenced herein, an “entity” can comprise a human, a client, a user, a computing device, a software application, an agent, a machine learning model, an artificial intelligence, and/or another entity. It should be appreciated that such an entity can facilitate implementation of the subject disclosure in accordance with one or more embodiments the described herein.

The computer processing systems, computer-implemented methods, apparatus and/or computer program products described herein employ hardware and/or software to solve problems that are highly technical in nature (e.g., determining and/or executing one or more actions determined to optimize vehicle battery health), that are not abstract and cannot be performed as a set of mental acts by a human.

In various embodiments, the health and/or vital signs of the driver can be monitored by a vehicle herein (e.g., a vehicle comprising autonomous driving capabilities). The foregoing can be achieved, for instance, by the vehicle, using the steering wheel comprising health monitoring sensors, connecting to wearable devices connected to the driver, etc. When the vehicle detects that driver is incapable of operating the vehicle, an autonomous drive system can be automatically activated. The activation can be triggered, for instance, while the vehicle is moving or stationary. If the vehicle is moving and a driver (or another vehicle occupant) suddenly becomes incapacitated (e.g., due to heart attack, stroke, etc.), the vehicle can activate an autonomous driving mode to take the occupant to safe place (e.g., a hospital, a home, or another defined location). In various embodiments, the vehicle can determine the shortest route to hospital with shortest time to treatment (e.g., shortest waiting time), alert other drivers/vehicles on the road to clear a path, and/or drive to the determined destination. The vehicle can alert a hospital, spouse, children, or any other defined emergency contact. In various embodiments, the vehicle can share its location or destination with one or more defined contacts or entities.

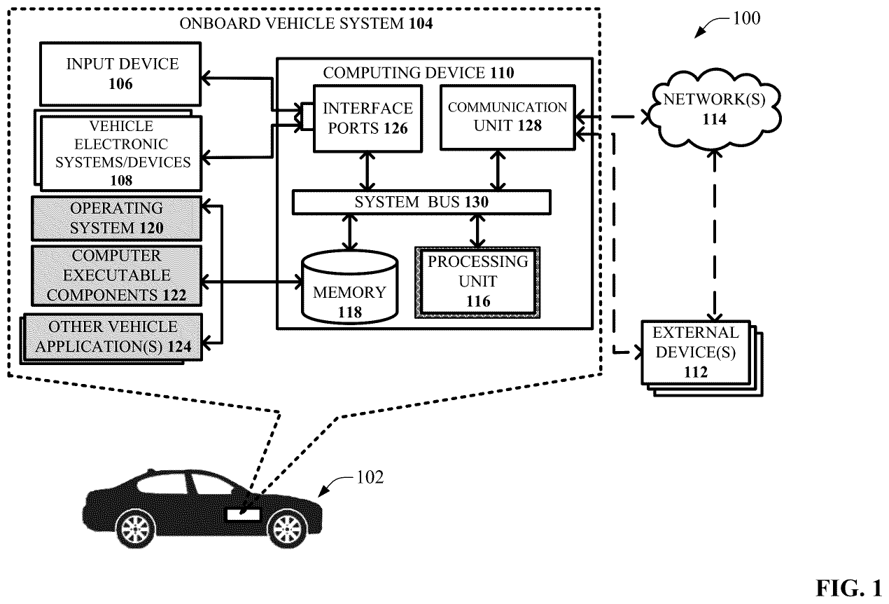

Turning now to FIG. 1, there is illustrated an example, non-limiting system 100 in accordance with one or more embodiments herein. System 100 can comprise a computerized tool, which can be configured to perform various operations relating to autonomous driving based on health of an occupant of a vehicle. In accordance with various exemplary embodiments, system 100 can be deployed on or within a vehicle 102, (e.g., an automobile, as shown in FIG. 1). Although FIG. 1 depicts the vehicle 102 as an automobile, the architecture of the system 100 is not so limited. For instance, the system 100 described herein can be implemented with a variety of types of vehicles 102. Example vehicles 102 that can incorporate the exemplary system 100 can include, but are not limited to: automobiles (e.g., autonomous vehicles), airplanes, trains, motorcycles, carts, trucks, semi-trucks, buses, boats, recreational vehicles, helicopters, jets, electric scooters, electric bicycles, vehicle chargers, vehicle charging stations, a combination thereof, and/or the like. It is additionally noted that the system 100 can be implemented in a variety of types of automobiles, such as battery electric vehicles, hybrid vehicles, plug-in hybrid vehicles, or other suitable types of vehicles.

As shown in FIG. 1, the system 100 can comprise one or more onboard vehicle systems 104, which can include one or more input devices 106, one or more other vehicle electronic systems and/or devices 108, and/or one or more computing devices 110. Additionally, the system 100 can comprise one or more external devices 112 that can be communicatively and/or operatively coupled to the one or more computing devices 110 of the one or more onboard vehicle systems 104 either via a one or more networks 114 and/or a direct electrical connection (e.g., as shown in FIG. 1). In various embodiments, one or more of the onboard vehicle system 104, input devices 106, vehicle electronic systems and/or devices 108, computing devices 110, external devices 112, and/or networks 114 can be communicatively or operably coupled (e.g., over a bus or wireless network) to one another to perform one or more functions of the system 100.

The one or more input devices 106 can display one or more interactive graphic entity interfaces (“GUIs”) that facilitate accessing and/or controlling various functions and/or application of the vehicle 102. The one or more input devices 106 can display one or more interactive GUIs that facilitate accessing and/or controlling various functions and/or applications. The one or more input devices 106 can comprise one or more computerized devices, which can include, but are not limited to: personal computers, desktop computers, laptop computers, cellular telephones (e.g., smartphones or mobile devices), computerized tablets (e.g., comprising a processor), smart watches, keyboards, touchscreens, mice, a combination thereof, and/or the like. An entity or user of the system 100 can utilize the one or more input devices 106 to input data into the system 100. Additionally, the one or more input devices 106 can comprise one or more displays that can present one or more outputs generated by the system 100 to an entity. For example, the one or more displays can include, but are not limited to: cathode tube display (“CRT”), light-emitting diode display (“LED”), electroluminescent display (“ELD”), plasma display panel (“PDP”), liquid crystal display (“LCD”), organic light-emitting diode display (“OLED”), a combination thereof, and/or the like.

For example, the one or more input devices 106 can comprise a touchscreen that can present one or more graphical touch controls that can respectively correspond to a control for a function of the vehicle 102, an application, a function of the application, interactive data, a hyperlink to data, and the like, wherein selection and/or interaction with the graphical touch control via touch activates the corresponding functionality. For instance, one or more GUIs displayed on the one or more input devices 106 can include selectable graphical elements, such as buttons or bars corresponding to a vehicle navigation application, a media application, a phone application, a back-up camera function, a car settings function, a parking assist function, and/or the like. In some implementations, selection of a button or bar corresponding to an application or function can result in the generation of a new window or GUI comprising additional selectable icons or widgets associated with the selected application. For example, selection of one or more selectable options herein can result in generation of a new GUI or window that includes additional buttons or widgets with one or more selectable options. The type and appearance of the controls can vary. For example, the graphical touch controls can include icons, symbols, widgets, windows, tabs, text, images, a combination thereof, and/or the like.

The one or more input devices 106 can comprise suitable hardware that registers input events in response to touch (e.g., by a finger, stylus, gloved hand, pen, etc.). In some implementations, the one or more input devices 106 can detect the position of an object (e.g., by a finger, stylus, gloved hand, pen, etc.) over the one or more input devices 106 within close proximity (e.g., a few centimeters) to touchscreen without the object touching the screen. As used herein, unless otherwise specified, reference to “on the touchscreen” refers to contact between an object (e.g., an entity's finger) and the one or more input devices 106 while reference to “over the touchscreen” refers to positioning of an object within close proximity to the touchscreen (e.g., a defined distance away from the touchscreen) yet not contacting the touchscreen.

The type of the input devices 106 can vary and can include, but is not limited to: a resistive touchscreen, a surface capacitive touchscreen, a projected capacitive touchscreen, a surface acoustic wave touchscreen, and an infrared touchscreen. In various embodiments, the one or more input devices 106 can be positioned on the dashboard of the vehicle 102, such as on or within the center stack or center console of the dashboard. However, the position of the one or more input devices 106 within the vehicle 102 can vary.

The one or more other vehicle electronic systems and/or devices 108 can include one or more additional devices and/or systems (e.g., in addition to the one or more input devices 106 and/or computing devices 110) of the vehicle 102 that can be controlled based at least in part on commands issued by the one or more computing devices 110 (e.g., via one or more processing units 116) and/or commands issued by the one or more external devices 112 communicatively coupled thereto. For example, the one or more other vehicle electronic systems and/or devices 108 can comprise: a media system (e.g., audio and/or video); a back-up camera system; a heating, ventilation, and air conditioning (“HVAC”) system; a lighting system; a cruise control system, a power locking system, a navigation system, an autonomous driving system, a vehicle sensor system, telecommunications system, a combination thereof, and/or the like. Other example other vehicle electronic systems and/or devices 108 can comprise one or more sensors, which can comprise odometers, altimeters, speedometers, accelerometers, engine features and/or components, fuel meters, flow meters, cameras (e.g., digital cameras, heat cameras, infrared cameras, and/or the like), lasers, radar systems, lidar systems, microphones, vibration meters, moisture sensors, thermometers, seatbelt sensors, wheel speed sensors, biometric and/or health sensors, a combination thereof, and/or the like. For instance, a speedometer of the vehicle 102 can detect the traveling speed of the vehicle 102. Further, the one or more sensors can detect and/or measure one or more conditions outside the vehicle 102, such as: whether the vehicle 102 is traveling through a rainy environment; whether the vehicle 102 is traveling through winter conditions (e.g., snowy and/or icy conditions); whether the vehicle 102 is traveling through very hot conditions (e.g., desert conditions); and/or the like. Example navigational information can include, but is not limited to: the destination of the vehicle 102, the position of the vehicle 102, the type of vehicle 102, the speed of the vehicle 102, environmental conditions surrounding the vehicle 102, the planned route of the vehicle 102, traffic conditions expected to be encountered by the vehicle 102, operational status of the vehicle 102, a combination thereof, and/or the like.

The one or more computing devices 110 can facilitate executing and controlling one or more operations of the vehicle 102, including one or more operations of the one or more input devices 106, and the one or more other vehicle electronic systems/devices 108 using machine-executable instructions. In this regard, embodiments of system 100 and other systems described herein can include one or more machine-executable components embodied within one or more machines (e.g., embodied in one or more computer readable storage media associated with one or more machines, such as computing device 110). Such components, when executed by the one or more machines (e.g., processors, computers, virtual machines, etc.) can cause the one or more machines to perform the operations described.

For example, the one or more computing devices 110 can include or be operatively coupled to at least one memory 118 and/or at least one processing unit 116. The one or more processing units 116 can be any of various available processors. For example, dual microprocessors and other multiprocessor architectures also can be employed as the processing unit 116. In various embodiments, the at least one memory 118 can store software instructions embodied as functions and/or applications that when executed by the at least one processing unit 116, facilitate performance of operations defined by the software instruction. In the embodiment shown, these software instructions can include one or more operating system 120, one or more computer-executable components 122, and/or one or more other vehicle applications 124. For example, the one or more operating systems 120 can act to control and/or allocate resources of the one or more computing devices 110. It is to be appreciated that the claimed subject matter can be implemented with various operating systems or combinations of operating systems.

The one or more computer executable components 122 and/or the one or more other vehicle applications 124 can take advantage of the management of resources by the one or more operating systems 120 through program modules and program data also stored in the one or more memories 118. The one or more computer executable components 122 can provide various features and/or functionalities that can facilitate autonomous driving based on health of an occupant of a vehicle herein. Example, other vehicle applications 124 can include, but are not limited to: a navigation application, a media player application, a phone application, a vehicle settings application, a parking assistance application, an emergency roadside assistance application, a combination thereof, and/or the like. The features and functionalities of the one or more computer executable components 122 are discussed in greater detail infra.

The one or more computing devices 110 can further include one or more interface ports 126, one or more communication units 128, and a system bus 130 that can communicatively couple the various features of the one or more computing devices 110 (e.g., the one or more interface ports 126, the one or more communication units 128, the one or more memories 118, and/or the one or more processing units 116). The one or more interface ports 126 can connect the one or more input devices 106 (and other potential devices) and the one or more other vehicle electronic systems/devices 108 to the one or more computing devices 110. For example, the one or more interface ports 126 can include, a serial port, a parallel port, a game port, a universal serial bus (“USB”) and the like.

The one or more communication units 128 can include suitable hardware and/or software that can facilitate connecting one or more external devices 112 to the one or more computing devices 110 (e.g., via a wireless connection and/or a wired connection). For example, the one or more communication units 128 can be operatively coupled to the one or more external devices 112 via one or more networks 114. The one or more networks 114 can include wired and/or wireless networks, including but not limited to, a personal area network (“PAN”), a local area network (“LAN”), a cellular network, a wide area network (“WAN”, e.g., the Internet), and the like. For example, the one or more external devices 112 can communicate with the one or more computing devices 110 (and vice versa) using virtually any desired wired or wireless technology, including but not limited to: wireless fidelity (“Wi-Fi”), global system for mobile communications (“GSM”), universal mobile telecommunications system (“UMTS”), worldwide interoperability for microwave access (“WiMAX”), enhanced general packet radio service (enhanced “GPRS”), fifth generation (“5G”) communication system, sixth generation (“6G”) communication system, third generation partnership project (“3GPP”) long term evolution (“LTE”), third generation partnership project 2 (“3GPP2”) ultra-mobile broadband (“UMB”), high speed packet access (“HSPA”), Zigbee and other 802.XX wireless technologies and/or legacy telecommunication technologies, near field communication (“NFC”) technology, BLUETOOTH®, Session Initiation Protocol (“SIP”), ZIGBEE®, RF4CE protocol, WirelessHART protocol, 6LoWPAN (IPv6 over Low power Wireless Area Networks), Z-Wave, an ANT, an ultra-wideband (“UWB”) standard protocol, vehicle-to-vehicle (V2V), vehicle-to-everything (V2X), and/or other proprietary and non-proprietary communication protocols. In this regard, the one or more communication units 128 can include software, hardware, or a combination of software and hardware that is configured to facilitate wired and/or wireless communication between the one or more computing devices 110 and the one or more external devices 112. While the one or more communication units 128 are shown for illustrative clarity as a separate unit that is not stored within memory 118, it is to be appreciated that one or more (software) components of the communication unit can be stored in memory 118 and include computer executable components.

The one or more external devices 112 can include any suitable computing device comprising a display and input device (e.g., a touchscreen) that can communicate with the one or more computing devices 110 comprised within the onboard vehicle system 104 and interface with the one or more computer executable components 122 (e.g., using a suitable application program interface (“API”)). For example, the one or more external devices 112 can include, but are not limited to: a mobile phone, a smartphone, a tablet personal computer (“PC”), a digital assistant (“PDA”), a heads-up display (“HUD”), virtual reality (“VR”) headset, an augmented reality (“AR”) headset, or another type of wearable computing device, a desktop computer, a laptop computer, a computer tablet, a combination thereof, and the like.

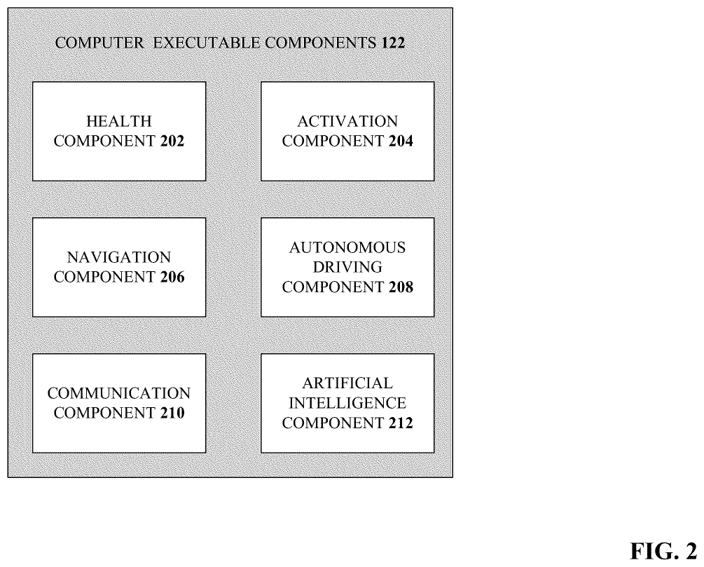

FIG. 2 illustrates a block diagram of example, non-limiting computer executable components 122 that can facilitate autonomous driving based on health of an occupant of a vehicle in accordance with one or more embodiments described herein. Repetitive description of like elements employed in other embodiments described herein is omitted for sake of brevity. As shown in FIG. 2, the one or more computer executable components 122 can include health component 202, activation component 204, navigation component 206, autonomous driving component 208, communication component 210, and/or artificial intelligence (A.I.) component 212.

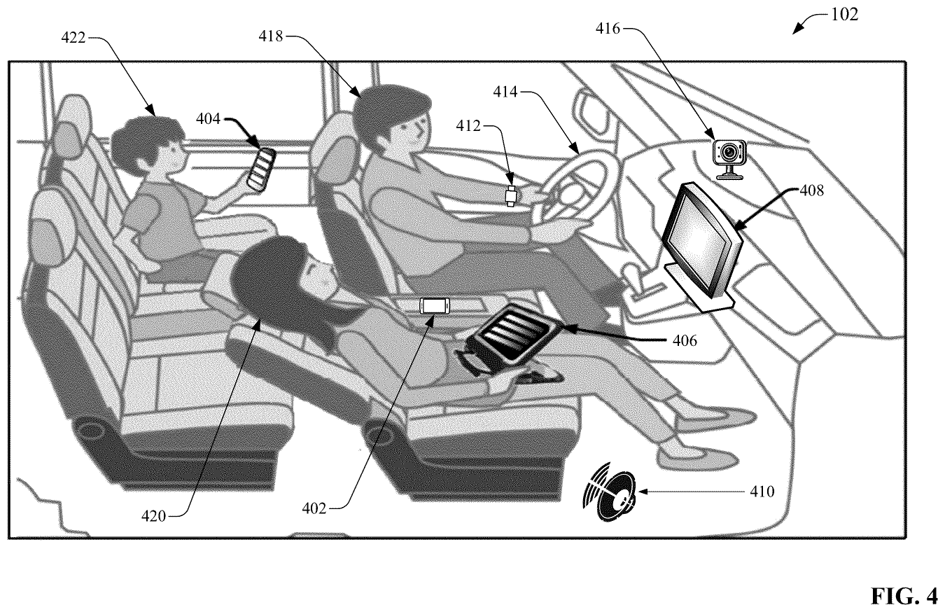

According to an embodiment, the health component 202 can, using a health sensor 312, determine one or more health metrics associated with an occupant of the vehicle 102, such as occupant 418 (e.g., a driver of the vehicle 102), occupant 420 (e.g., a passenger of the vehicle 102), and/or occupant 422 (e.g., a passenger of the vehicle 102). It is noted that while three occupants are depicted in FIG. 4, embodiments herein are not so limited, and any quantity of monitorable occupants is envisaged. The health metric herein can vary, for instance, depending on the type of health sensor 312 utilized by the health component 202. For instance, in various embodiments, the health sensor 312 can comprise a sensor of a steering wheel 414 of the vehicle 102. In this regard, contact between a hand of an occupant and the steering wheel 414 can enable the health sensor 312 to gather and/or determine one or more health metrics herein. In further embodiments, the health sensor 312 can comprise a camera 416 in an interior of the vehicle 102. In this regard, the camera 416 can monitor an occupant's facial expressions and/or body posture. For example, a slumped or unresponsive posture of an occupant of a vehicle 102 herein can suggest incapacitation of that occupant of the vehicle 102. In various embodiments, the camera 416 can further track eyes of an occupant of the vehicle 102, for instance, to detect if the occupant's eyes are closed for an extended period, which can indicate unconsciousness of the occupant of the vehicle 102.

In additional embodiments, the health sensor 312 can comprise a wearable device 412 (e.g., smart watch, activity/fitness tracker, skin patches with embedded sensors, smart clothing or integrated activewear, smart contact lenses or glasses, smart rings, smart jewelry, smart bandages, or other suitable wearable devices), worn by the occupant of the vehicle 102, and communicatively coupled (e.g., via the communication component 210) to the vehicle 102. Thus, the health component 202 can, using the wearable device 412, determine one or more health metrics associated with an occupant of the vehicle 102. In further embodiments, the health sensor 312 can comprise an electrocardiogram sensor, a pulse oximeter sensor, a glucose monitoring sensor, respiration sensor, seat pressure sensor, infrared sensor, and/or another suitable health sensor 312.

According to an embodiment, the activation component 204 can, based on a defined activation criterion associated with the one or more health metrics being determined to be satisfied (e.g., via the activation component 204), activate an autonomous driving mode (e.g., of the vehicle 102). In various embodiments, the autonomous driving mode can comprise Society of Automotive Engineers (SAE) level 4 or level 5 autonomous driving, or another suitable autonomous driving level. In various embodiments, the defined activation criterion herein can comprise threshold likely incapacitation of the occupant of the vehicle 102, (e.g., as determined via the health component 202). In this regard, the health component 202 can determine a probability, based on the one or more health metrics, that the occupant of the vehicle 102 is incapacitated, and if the probability satisfies a defined threshold, the activation component 204 can activate the autonomous driving mode of the vehicle 102. In various embodiments, the A.I. component 212 can determine the likelihood of incapacitation of the occupant of the vehicle 102 using machine learning applied to prior instances of prior occupants of vehicles and/or incapacitation of said prior occupants. In this regard, determinations of incapacitations of occupants herein can be refined and improved over time based on feedback regarding the accuracy of such determinations.

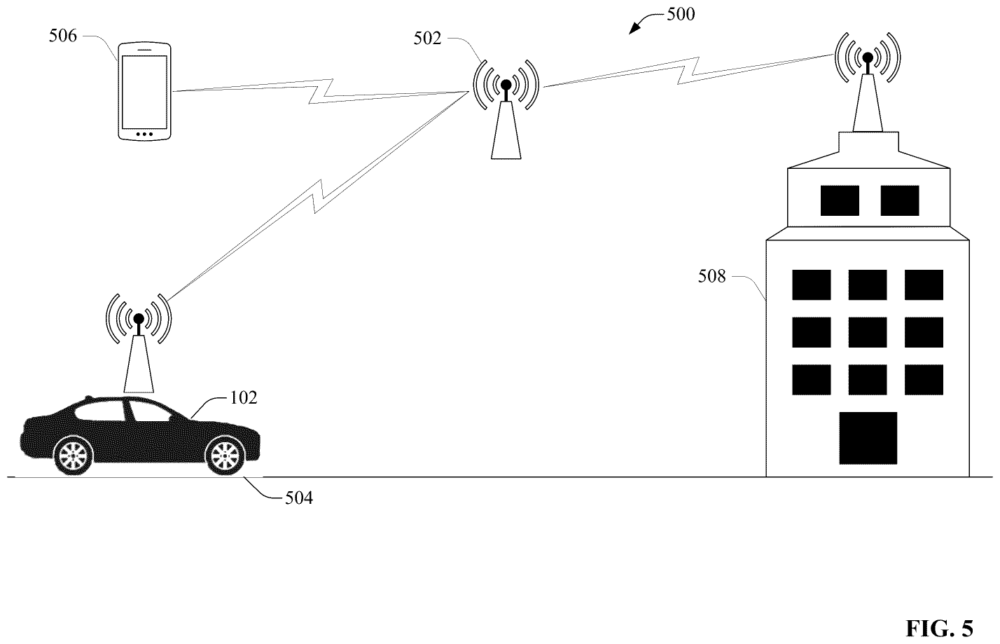

According to an embodiment, the navigation component 206 can, in response to activation of the autonomous driving mode, determine a route from a current location of the vehicle to a defined location (e.g., a destination). In this regard, the navigation component 206 can process the current location (e.g., current location 504 in FIG. 5) and the defined location (e.g., defined location 508 in FIG. 5), considering factors in-between such as road types, traffic conditions (e.g., if available), and/or any user preferences. In various embodiments, the navigation component 206 can calculate the optimal route (e.g., from the current location 504 to the defined location 508) based on such information. In various embodiments, the A.I. component 212 can determine optimal routes for the vehicle 102 using machine learning applied to past routes of other vehicles, other than the vehicle 102, and/or past routes of the vehicle 102. In various embodiments, the defined location 508 herein can be determined (e.g., via the navigation component 206 and/or A.I. component 212) based on the one or more health metrics determined via the health component 202. For example, if the health component 202 determines that the occupant of the vehicle 102 is having a heart attack, the defined location can be set (e.g., via the navigation component 206 and/or A.I. component 212) as a hospital capable of treating such patients. In another example, if the health component 202 determines that the occupant of the vehicle 102 is merely tired and needs to sleep, the defined location can be set (e.g., via the navigation component 206 and/or A.I. component 212) as a home associated with said occupant of the vehicle 102. In various embodiments, the route can be determined to comprise a shortest distance from the current location 504 the defined location 508. In further embodiments, the route can be determined (e.g., via the navigation component 206 and/or A.I. component 212) to comprise a shortest amount of travel time between the current location 504 and the defined location 508. In additional embodiments, the route can be determined (e.g., via the navigation component 206 and/or A.I. component 212) based on a predicted treatment time at a group of defined locations, comprising the defined location 508. For example, if the health component 202 determines that the occupant of the vehicle 102 is having a heart attack, the navigation component 206 and/or A.I. component 212 can determine a plurality of hospitals capable of treating such patients. In addition to travel distance or travel time, the navigation component 206 and/or A.I. component 212 can also determine expected wait times at the plurality of hospitals by communicating (e.g., via the communication component 210) with said plurality of hospitals, and then factoring in such wait times along with travel time in order to determine the fastest care possible for the occupant of the vehicle 102.

According to an embodiment, the autonomous driving component 208 can, using the route determined via the navigation component 206 and/or the A.I. component 212, autonomously navigate the vehicle 102 to the defined location 508. In this regard, the autonomous driving component 208 can utilize a combination of sensor data, for instance, to safely and efficiently drive the vehicle 102 without occupant intervention. Vehicle sensors, such as LiDAR, radar, cameras, ultrasonic sensors, or other suitable sensors (e.g., of the vehicle electronic systems/devices 108) can be utilized (e.g., via the autonomous driving component 208) to perceive surroundings of the vehicle 102. In various embodiments, the autonomous driving component 208 can continuously collect data on nearby objects, road conditions, and traffic, enabling the autonomous driving component 208 to create a real-time map of the environment surrounding the vehicle 102. In various embodiments, the A.I. component 212 can processes this data to make driving decisions, including path planning, obstacle avoidance, and/or speed control applicable to the vehicle 102. In various embodiments, the autonomous driving component 208 can guide the vehicle 102 by controlling steering, acceleration, and/or braking of the vehicle 102. In various embodiments, the autonomous driving component 208 can continuously update its decisions based on changing road conditions, ensuring safety and efficiency throughout the journey from the current location 504 to the defined location 508 (e.g., a hospital, urgent care facility, medical facility, house, or another suitable defined location).

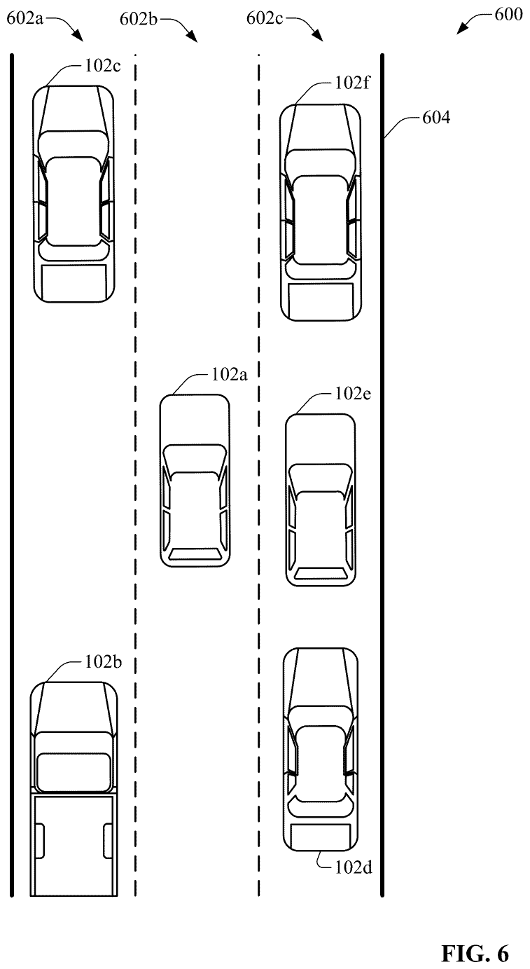

According to an embodiment, the communication component 210 can transmit, to other vehicles (e.g., vehicles 102b, 102c, 102d, 102e, and/or 102f) on a route (e.g., determined via the navigation component 206 and/or A.I. component 212) (e.g., on a road 604 shared with the vehicle 102a), other than the vehicle 102a, an instruction to clear a path (e.g., a lane) for the vehicle 102a along the route (see, e.g., FIG. 6). It is noted that, in various embodiments, vehicles 102a, 102b, 102c, 102d, 102e, and/or 102f can be similar to the vehicle 102 and/or comprise like components and functionality. In FIG. 6, vehicle 102c can, based on the instruction to clear the path for the vehicle 102a, move from lane 602b to lane 602a, and vehicle 102f can, based on the instruction to clear the path for the vehicle 102a, move from lane 602b to lane 602c, for instance, in order to clear a path for the vehicle 102a to travel to the defined location 508 as quickly and safely possible. In various embodiments, the vehicle 102a can transmit a distress signal or emergency message to nearby vehicles (e.g., vehicles 102b, 102c, 102d, 102e, and/or 102f) and/or traffic management systems through a V2V and/or V2X communication protocol. The distress signal or emergency message can comprise information regarding the emergency and/or the need for priority clearance. In further embodiments, the vehicle 102a can additionally utilize emergency lights (e.g., flashing lights) of the vehicle 102a, and/or a siren or horn of the vehicle 102a in order to communicate the emergency nature of the occupant of the vehicle 102a. In various embodiments, nearby vehicles (e.g., vehicles 102b, 102c, 102d, 102e, and/or 102f receiving the emergency message from the vehicle 102a can adapt their respective behaviors, such as slowing down, changing lanes, or pulling over to allow the vehicle 102a to pass safely and quickly.

According to an embodiment, the communication component 210 can transmit an alert, associated with the activation of the autonomous driving mode (e.g., via the activation component 204) or the one or more health metrics, to the defined location (e.g., defined location 508). For example, if the health component 202 determines that the occupant of the vehicle 102 is having a heart attack, the communication component 210 can transmit various cardiovascular metric(s) captured via the health sensor 312 to a destination hospital (e.g., defined location 508). In further embodiments, the communication component 210 can transmit an alert, associated with the activation (e.g., via the activation component 204) of the autonomous driving mode or the one or more health metrics, to a registered recipient (e.g., comprising or associated with a mobile device 506 registered with the occupant of the vehicle 102) associated with the occupant of the vehicle 102. In this regard, a user/occupant of the vehicle 102 can register emergency contacts to be automatically contacted (e.g., via the communication component 210) in response to activation (e.g., via the activation component 204) of the autonomous driving mode of the vehicle 102. The alert to the registered recipient(s) can further comprise corresponding health metrics applicable to the occupant of the vehicle 102 that has the health emergency. In various embodiments, the alert can comprise the current location 504 and the defined location 508. In one or more embodiments, the communication component 210 can send updated current locations 504 as the vehicle 102 travels to the defined location 508. In various implementations, such an alert can comprise a push notification (e.g., via a mobile application of a mobile device 506), a text message, an email, a phone call, a video message, voicemail, or another suitable message. In additional embodiments, an alert herein can be transmitted via an infotainment device 408 of the vehicle 102 (e.g., to alert other occupants of the vehicle 102 regarding an incapacitation of an occupant of the vehicle 102 and/or to prompt the occupant to prevent activation of an autonomous driving mode, for instance, if the activation is a false alarm). In further embodiments, the message can comprise a haptic alert via a steering wheel 414 of the vehicle 102, an audible alert via a speaker 410 of the vehicle 102, or a different type of message, and the user can provide an audible response (e.g., captured via a microphone of the vehicle 102). In various implementations, the message can increase in intensity over a defined period of time (e.g., via haptic vibration or sound) in response to the message not being acknowledged by the user (e.g., via a corresponding mobile device 402, 404, 406, or 506, infotainment device 408, or via another suitable interface). It is noted that, in various embodiments, the message can be transmitted to/from an external device (e.g., a mobile device 402, 404, 406, and/or 506) and a vehicle 102 via a V2V and/or V2X communication protocol.

Various embodiments herein can employ artificial-intelligence or machine learning systems and techniques to facilitate learning user behavior, context-based scenarios, preferences, etc. in order to facilitate taking automated action with high degrees of confidence. Utility-based analysis can be utilized to factor benefit of taking an action against cost of taking an incorrect action. Probabilistic or statistical-based analyses can be employed in connection with the foregoing and/or the following.

It is noted that systems and/or associated controllers, servers, or machine learning components herein can comprise artificial intelligence component(s) which can employ an artificial intelligence (A.I.) model and/or machine learning (M.L.) or an M.L. model that can learn to perform the above or below described functions (e.g., via training using historical training data and/or feedback data).

In some embodiments, A.I. component 212 can comprise an A.I. and/or M.L. model that can be trained (e.g., via supervised and/or unsupervised techniques) to perform the above or below-described functions using historical training data comprising various context conditions that correspond to various augmented network optimization operations. In this example, such an A.I. and/or M.L. model can further learn (e.g., via supervised and/or unsupervised techniques) to perform the above or below-described functions using training data comprising feedback data, where such feedback data can be collected and/or stored (e.g., in memory) by the A.I. component 212. In this example, such feedback data can comprise the various instructions described above/below that can be input, for instance, to a system herein, over time in response to observed/stored context-based information.

A.I./M.L. components herein can initiate an operation(s) associated with a based on a defined level of confidence determined using information (e.g., feedback data). For example, based on learning to perform such functions described above using feedback data, performance information, and/or past performance information herein, a A.I. component 212 herein can initiate an operation associated with determining various thresholds herein (e.g., a motion pattern thresholds, input pattern thresholds, similarity thresholds, authentication signal thresholds, audio frequency thresholds, or other suitable thresholds).

In an embodiment, the A.I. component 212 can perform a utility-based analysis that factors cost of initiating the above-described operations versus benefit. In this embodiment, the A.I. component 212 can use one or more additional context conditions to determine various thresholds herein.

To facilitate the above-described functions, a A.I. component 212 herein can perform classifications, correlations, inferences, and/or expressions associated with principles of artificial intelligence. For instance, the A.I. component 212 can employ an automatic classification system and/or an automatic classification. In one example, the A.I. component 212 can employ a probabilistic and/or statistical-based analysis (e.g., factoring into the analysis utilities and costs) to learn and/or generate inferences. The A.I. component 212 can employ any suitable machine-learning based techniques, statistical-based techniques and/or probabilistic-based techniques. For example, the A.I. component 212 can employ expert systems, fuzzy logic, support vector machines (SVMs), Hidden Markov Models (HMMs), greedy search algorithms, rule-based systems, Bayesian models (e.g., Bayesian networks), neural networks, other non-linear training techniques, data fusion, utility-based analytical systems, systems employing Bayesian models, and/or the like. In another example, the A.I. component 212 can perform a set of machine-learning computations. For instance, the A.I. component 212 can perform a set of clustering machine learning computations, a set of logistic regression machine learning computations, a set of decision tree machine learning computations, a set of random forest machine learning computations, a set of regression tree machine learning computations, a set of least square machine learning computations, a set of instance-based machine learning computations, a set of regression machine learning computations, a set of support vector regression machine learning computations, a set of k-means machine learning computations, a set of spectral clustering machine learning computations, a set of rule learning machine learning computations, a set of Bayesian machine learning computations, a set of deep Boltzmann machine computations, a set of deep belief network computations, and/or a set of different machine learning computations.

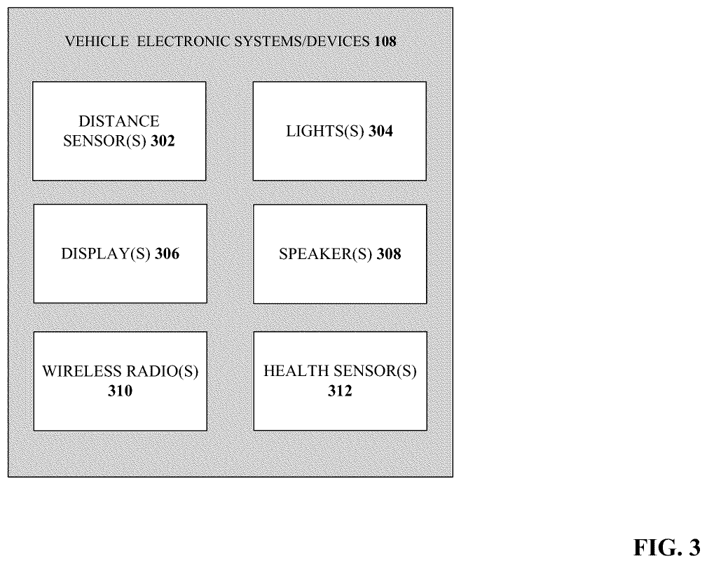

FIG. 3 illustrates a block diagram of example, non-limiting vehicle electronic systems/devices 108 in accordance with one or more embodiments described herein. Repetitive description of like elements employed in other embodiments described herein is omitted for sake of brevity. In various embodiments, the vehicle electronic systems/devices 108 can comprise one or more of distance sensors 302 (e.g., sensors external or internal to the vehicle 102 that are arranged to detect distance between the vehicle 102 and other objects (e.g., other vehicles, pedestrians, etc.) and/or to determine potential collisions, such as one or more of a lidar sensor, a radar sensor, an ultrasonic sensor, an infrared sensor, a laser sensor, a light emitting diode (LED) sensor, a capacitive sensor, a time of flight sensor, a hall effect sensor, or an optical sensor, or another suitable sensors), light(s) 304, display(s) 306 (e.g., infotainment devices, touchscreen displays, etc.), speaker(s) 308, and/or wireless radio(s) 310. In various embodiments, the vehicle electronic systems/devices 108 can further comprise one or more health sensors 312 described herein.

FIG. 4 illustrates a diagram of an example, non-limiting vehicle 102 interior in accordance with one or more embodiments described herein. FIG. 4 depicts mobile devices 402, 404, and 406, infotainment device 408, speaker 410, wearable device 412 (e.g., a smartwatch worn by occupant 418), steering wheel 414 of the vehicle 102, camera 416 of the vehicle 102, occupant 418 (e.g., a driver of the vehicle 102), occupant 420 (e.g., a front passenger of the vehicle 102), and occupant 422 (e.g., a rear passenger of the vehicle 102). In various embodiments herein, the health sensor 312 can comprise a sensor of a steering wheel 414 of the vehicle 102. In this regard, contact between a hand of an occupant and the steering wheel 414 can enable the health sensor 312 to gather and/or determine one or more health metrics herein. In various embodiments, the health sensor 312 can comprise a camera 416 in an interior of the vehicle 102. In this regard, the camera 416 can monitor an occupant's facial expressions and/or body posture. For example, a slumped or unresponsive posture of an occupant of a vehicle 102 herein can suggest incapacitation of that occupant of the vehicle 102. In various embodiments, the camera 416 can further track eyes of an occupant of the vehicle 102, for instance, to detect if the occupant's eyes are closed for an extended period, which can indicate unconsciousness of the occupant of the vehicle 102.

FIG. 5 illustrates an exemplary scenario 500 in accordance with one or more embodiments described herein. In scenario 500, the vehicle 102, at a current location 504, can be communicatively connected (e.g., via the communication component 210) to a mobile device 506 and/or a defined location 508 (e.g., a building such as a hospital, medical facility, urgent care facility, home, or other suitable defined location) via a cellular connection facilitated via the cellular tower 502. In further embodiments, the vehicle 102 can communicate directly (e.g., via the communication component 210) with the mobile device 506 and/or the defined location 508. In various embodiments, the mobile device 506 can comprise a registered recipient (e.g., of a friend or family) associated with the occupant of the vehicle 102. In this regard, a user/occupant of the vehicle 102 can register emergency contacts to be automatically contacted (e.g., via the communication component 210) in response to activation (e.g., via the activation component 204) of the autonomous driving mode of the vehicle 102. The alert to the registered recipient(s) can further comprise corresponding health metrics applicable to the occupant of the vehicle 102 that has the health emergency. In various embodiments, the alert can comprise the current location 504 and the defined location 508. In one or more embodiments, the communication component 210 can send updated current locations 504 as the vehicle 102 travels to the defined location 508. In various implementations, such an alert can comprise a push notification (e.g., via a mobile application of a mobile device 506), a text message, an email, a phone call, a video message, voicemail, or another suitable message.

FIG. 6 illustrates an exemplary scenario 600 in accordance with one or more embodiments described herein. In scenario 600, vehicles 102c can move from lane 602b to lane 602a, and vehicle 102f can move from lane 602b to lane 602c in order to clear a path for the vehicle 102a to travel to the defined location as quickly as safely possible (e.g., based on an instruction sent from the vehicle 102a via the communication component 210 of the vehicle 102a). In various embodiments, the vehicle 102a can transmit (e.g., via the communication component 210) a distress signal or emergency message to nearby vehicles (e.g., vehicles 102b, 102c, 102d, 102e, and/or 102f) and/or traffic management systems through V2V and/or V2X communication. It is noted that, in various embodiments, vehicles 102a, 102b, 102c, 102d, 102e, and/or 102f can be similar to the vehicle 102 and/or comprise like components and functionality. In various embodiments, vehicle 102c can, based on an instruction to clear the path for the vehicle 102a, move from lane 602b to lane 602a, and vehicle 102f can, based on the instruction to clear the path for the vehicle 102a, move from lane 602b to lane 602c, for instance, in order to clear a path for the vehicle 102a to travel to the defined location 508 as quickly and safely possible. In various embodiments, the vehicle 102a can transmit a distress signal or emergency message to nearby vehicles (e.g., vehicles 102b, 102c, 102d, 102e, and/or 102f) and/or traffic management systems through a V2V and/or V2X communication protocol. The distress signal or emergency message can comprise information regarding the emergency and/or the need for priority clearance. In further embodiments, the vehicle 102a can additionally utilize emergency lights (e.g., flashing lights) of the vehicle 102a, and/or a siren or horn of the vehicle 102a in order to communicate the emergency nature of the occupant of the vehicle 102a. In various embodiments, nearby vehicles (e.g., vehicles 102b, 102c, 102d, 102e, and/or 102f receiving the emergency message from the vehicle 102a can adapt their respective behaviors, such as slowing down, changing lanes, or pulling over to allow the vehicle 102a to pass safely and quickly.

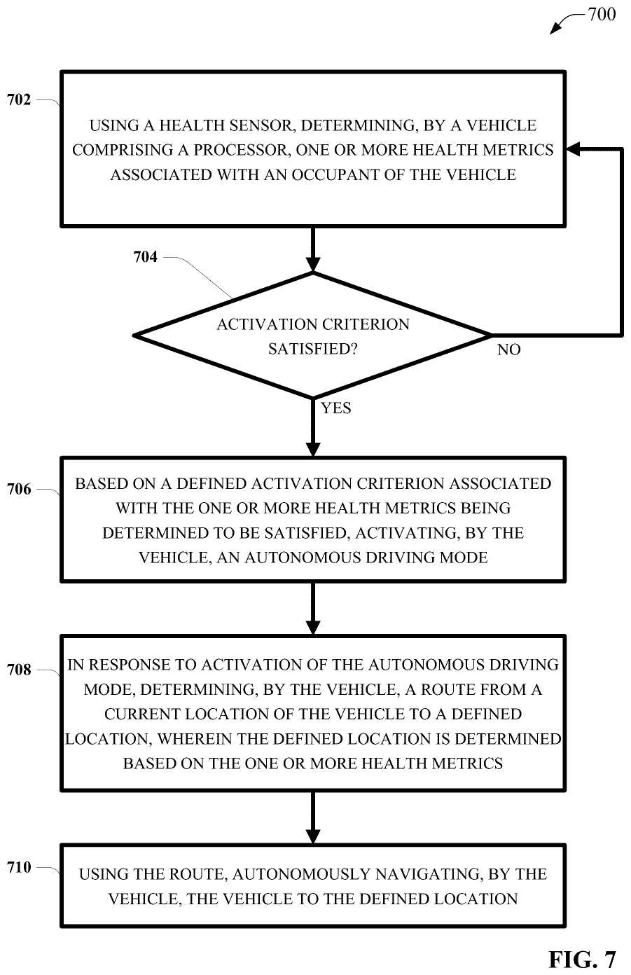

FIG. 7 illustrates a block flow diagram for a process 700 associated with autonomous driving based on health of an occupant of a vehicle in accordance with one or more embodiments described herein. At 702, the process 700 can comprise, using a health sensor (e.g., health sensor 312), determining (e.g., via the health component 202), by a vehicle (e.g., vehicle 102) comprising a processor, one or more health metrics associated with an occupant of the vehicle (e.g., vehicle 102). At 704, if an activation criterion is determined (e.g., via the activation component 204) to be satisfied (e.g., YES at 704), the process 700 can proceed to 706. If at 704, the activation criterion is determined (e.g., via the activation component 204) not to be satisfied (e.g., NO at 704), the process 700 can return to 702. At 706, the process 700 can comprise, based on a defined activation criterion associated with the one or more health metrics being determined (e.g., via the activation component 204) to be satisfied, activating (e.g., via the activation component 204), by the vehicle (e.g., vehicle 102), an autonomous driving mode. At 708, the process 700 can comprise, in response to activation (e.g., via the activation component 204) of the autonomous driving mode, determining (e.g., via the navigation component 206), by the vehicle (e.g., vehicle 102), a route from a current location (e.g., current location 504) of the vehicle (e.g., vehicle 102) to a defined location (e.g., defined location 508), wherein the defined location (e.g., defined location 508) is determined (e.g., via the navigation component 206 and/or A.I. component 212) based on the one or more health metrics. At 710, the process 700 can comprise, using the route, autonomously navigating (e.g., via the autonomous driving component 208 and/or A.I. component 212), by the vehicle (e.g., vehicle 102), the vehicle (e.g., vehicle 102) to the defined location (e.g., defined location 508).

Systems described herein can be coupled (e.g., communicatively, electrically, operatively, optically, inductively, acoustically, etc.) to one or more local or remote (e.g., external) systems, sources, and/or devices (e.g., electronic control systems (ECU), classical and/or quantum computing devices, communication devices, etc.). For example, system 100 (or other systems, controllers, processors, etc.) can be coupled (e.g., communicatively, electrically, operatively, optically, etc.) to one or more local or remote (e.g., external) systems, sources, and/or devices using a data cable (e.g., High-Definition Multimedia Interface (HDMI), recommended standard (RS), Ethernet cable, etc.) and/or one or more wired networks described below.

In some embodiments, systems herein can be coupled (e.g., communicatively, electrically, operatively, optically, inductively, acoustically, etc.) to one or more local or remote (e.g., external) systems, sources, and/or devices (e.g., electronic control units (ECU), classical and/or quantum computing devices, communication devices, etc.) via a network. In these embodiments, such a network can comprise one or more wired and/or wireless networks, including, but not limited to, a cellular network, a wide area network (WAN) (e.g., the Internet), and/or a local area network (LAN). For example, system 100 can communicate with one or more local or remote (e.g., external) systems, sources, and/or devices, for instance, computing devices using such a network, which can comprise virtually any desired wired or wireless technology, including but not limited to: powerline ethernet, VHF, UHF, AM, wireless fidelity (Wi-Fi), BLUETOOTH®, fiber optic communications, global system for mobile communications (GSM), universal mobile telecommunications system (UMTS), worldwide interoperability for microwave access (WiMAX), enhanced general packet radio service (enhanced GPRS), third generation partnership project (3GPP) long term evolution (LTE), third generation partnership project 2 (3GPP2) ultra-mobile broadband (UMB), high speed packet access (HSPA), Zigbee and other 802.XX wireless technologies and/or legacy telecommunication technologies, Session Initiation Protocol (SIP), ZIGBEE®, RF4CE protocol, WirelessHART protocol, L-band voice or data information, 6LoWPAN (IPv6 over Low power Wireless Area Networks), Z-Wave, an ANT, an ultra-wideband (UWB) standard protocol, and/or other proprietary and non-proprietary communication protocols. In this example, system 100 can thus include hardware (e.g., a central processing unit (CPU), a transceiver, a decoder, an antenna (e.g., a ultra-wideband (UWB) antenna, a BLUETOOTH® low energy (BLE) antenna, etc.), quantum hardware, a quantum processor, etc.), software (e.g., a set of threads, a set of processes, software in execution, quantum pulse schedule, quantum circuit, quantum gates, etc.), or a combination of hardware and software that facilitates communicating information between a system herein and remote (e.g., external) systems, sources, and/or devices (e.g., computing and/or communication devices such as, for instance, a smart phone, a smart watch, wireless earbuds, etc.).

System herein can comprise one or more computer and/or machine readable, writable, and/or executable components and/or instructions that, when executed by processor (e.g., a processing unit 116 which can comprise a classical processor, a quantum processor, etc.), can facilitate performance of operations defined by such component(s) and/or instruction(s). Further, in numerous embodiments, any component associated with a system herein, as described herein with or without reference to the various figures of the subject disclosure, can comprise one or more computer and/or machine readable, writable, and/or executable components and/or instructions that, when executed by a processor, can facilitate performance of operations defined by such component(s) and/or instruction(s). Consequently, according to numerous embodiments, system herein and/or any components associated therewith as disclosed herein, can employ a processor (e.g., processing unit 116) to execute such computer and/or machine readable, writable, and/or executable component(s) and/or instruction(s) to facilitate performance of one or more operations described herein with reference to system herein and/or any such components associated therewith.

Systems herein can comprise any type of system, device, machine, apparatus, component, and/or instrument that comprises a processor and/or that can communicate with one or more local or remote electronic systems and/or one or more local or remote devices via a wired and/or wireless network. All such embodiments are envisioned. For example, a system (e.g., a system 100 or any other system or device described herein) can comprise a computing device, a general-purpose computer, field-programmable gate array, AI accelerator application-specific integrated circuit, a special-purpose computer, an onboard computing device, a communication device, an onboard communication device, a server device, a quantum computing device (e.g., a quantum computer), a tablet computing device, a handheld device, a server class computing machine and/or database, a laptop computer, a notebook computer, a desktop computer, wearable device, internet of things device, a cell phone, a smart phone, a consumer appliance and/or instrumentation, an industrial and/or commercial device, a digital assistant, a multimedia Internet enabled phone, a multimedia players, and/or another type of device.

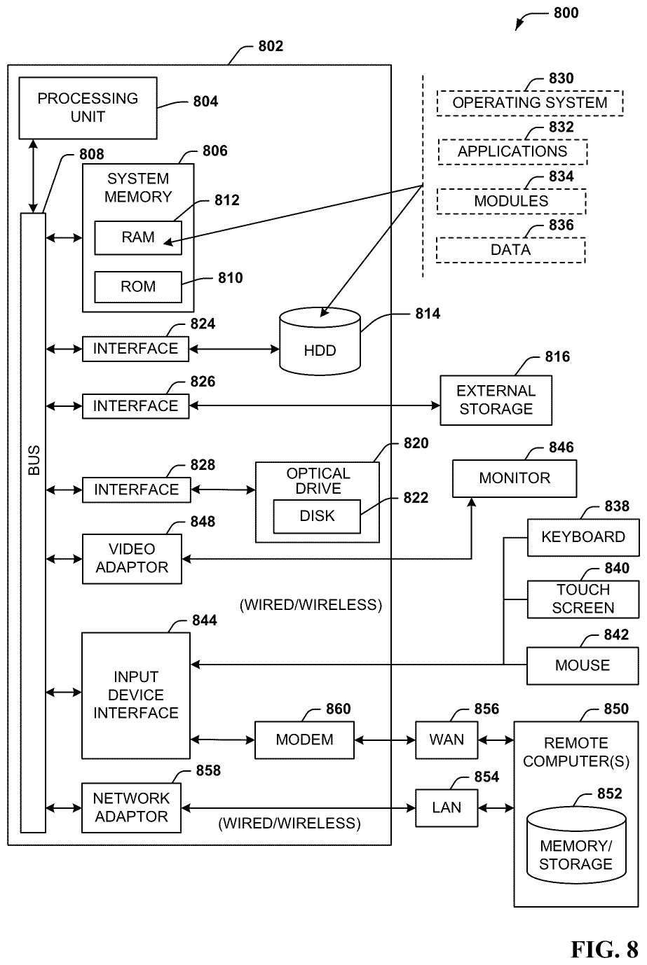

In order to provide additional context for various embodiments described herein, FIG. 8 and the following discussion are intended to provide a brief, general description of a suitable computing environment 800 in which the various embodiments of the embodiment described herein can be implemented. While the embodiments have been described above in the general context of computer-executable instructions that can run on one or more computers, those skilled in the art will recognize that the embodiments can be also implemented in combination with other program modules and/or as a combination of hardware and software.

Generally, program modules include routines, programs, components, data structures, etc., that perform particular tasks or implement particular abstract data types. Moreover, those skilled in the art will appreciate that the various methods can be practiced with other computer system configurations, including single-processor or multiprocessor computer systems, minicomputers, mainframe computers, Internet of Things (IoT) devices, distributed computing systems, as well as personal computers (e.g., ruggedized personal computers), field-programmable gate arrays, hand-held computing devices, microprocessor-based or programmable consumer electronics, and the like, each of which can be operatively coupled to one or more associated devices.

The illustrated embodiments of the embodiments herein can be also practiced in distributed computing environments where certain tasks are performed by remote processing devices that are linked through a communications network. In a distributed computing environment, program modules can be located in both local and remote memory storage devices.

Computing devices typically include a variety of media, which can include computer-readable storage media, machine-readable storage media, and/or communications media, which two terms are used herein differently from one another as follows. Computer-readable storage media or machine-readable storage media can be any available storage media that can be accessed by the computer and includes both volatile and nonvolatile media, removable and non-removable media. By way of example, and not limitation, computer-readable storage media or machine-readable storage media can be implemented in connection with any method or technology for storage of information such as computer-readable or machine-readable instructions, program modules, structured data, or unstructured data.

Computer-readable storage media can include, but are not limited to, random access memory (RAM), read only memory (ROM), electrically erasable programmable read only memory (EEPROM), flash memory or other memory technology, compact disk read only memory (CD ROM), digital versatile disk (DVD), Blu-ray disc (BD) or other optical disk storage, magnetic cassettes, magnetic tape, magnetic disk storage or other magnetic storage devices, solid state drives or other solid state storage devices, or other tangible and/or non-transitory media which can be used to store desired information. In this regard, the terms “tangible” or “non-transitory” herein as applied to storage, memory, or computer-readable media, are to be understood to exclude only propagating transitory signals per se as modifiers and do not relinquish rights to all standard storage, memory or computer-readable media that are not only propagating transitory signals per se.

Computer-readable storage media can be accessed by one or more local or remote computing devices, e.g., via access requests, queries, or other data retrieval protocols, for a variety of operations with respect to the information stored by the medium.

Communications media typically embody computer-readable instructions, data structures, program modules or other structured or unstructured data in a data signal such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, optic, infrared, and other wireless media.

With reference again to FIG. 8, the example environment 800 for implementing various embodiments of the aspects described herein includes a computer 802, the computer 802 including a processing unit 804, a system memory 806 and a system bus 808. The system bus 808 couples system components including, but not limited to, the system memory 806 to the processing unit 804. The processing unit 804 can be any of various commercially available processors, field-programmable gate array, AI accelerator application-specific integrated circuit, or other suitable processors. Dual microprocessors and other multi-processor architectures can also be employed as the processing unit 804.

The system bus 808 can be any of several types of bus structure that can further interconnect to a memory bus (with or without a memory controller), a peripheral bus, and a local bus using any of a variety of commercially available bus architectures. The system memory 806 includes ROM 810 and RAM 812. A basic input/output system (BIOS) can be stored in a nonvolatile memory such as ROM, erasable programmable read only memory (EPROM), EEPROM, which BIOS contains the basic routines that help to transfer information between elements within the computer 802, such as during startup. The RAM 812 can also include a high-speed RAM such as static RAM for caching data. It is noted that unified Extensible Firmware Interface(s) can be utilized herein.

The computer 802 further includes an internal hard disk drive (HDD) 814 (e.g., EIDE, SATA), one or more external storage devices 816 (e.g., a magnetic floppy disk drive (FDD) 816, a memory stick or flash drive reader, a memory card reader, etc.) and an optical disk drive 820 (e.g., which can read or write from a disk 822 such as a CD-ROM disc, a DVD, a BD, etc.). While the internal HDD 814 is illustrated as located within the computer 802, the internal HDD 814 can also be configured for external use in a suitable chassis (not shown). Additionally, while not shown in environment 800, a solid-state drive (SSD) could be used in addition to, or in place of, an HDD 814. The HDD 814, external storage device(s) 816 and optical disk drive 820 can be connected to the system bus 808 by an HDD interface 824, an external storage interface 826 and an optical drive interface 828, respectively. The interface 824 for external drive implementations can include at least one or both of Universal Serial Bus (USB) and Institute of Electrical and Electronics Engineers (IEEE) 1394 interface technologies. Other external drive connection technologies are within contemplation of the embodiments described herein.

The drives and their associated computer-readable storage media provide nonvolatile storage of data, data structures, computer-executable instructions, and so forth. For the computer 802, the drives and storage media accommodate the storage of any data in a suitable digital format. Although the description of computer-readable storage media above refers to respective types of storage devices, it should be appreciated by those skilled in the art that other types of storage media which are readable by a computer, whether presently existing or developed in the future, could also be used in the example operating environment, and further, that any such storage media can contain computer-executable instructions for performing the methods described herein.

A number of program modules can be stored in the drives and RAM 812, including an operating system 830, one or more application programs 832, other program modules 834 and program data 836. All or portions of the operating system, applications, modules, and/or data can also be cached in the RAM 812. The systems and methods described herein can be implemented utilizing various commercially available operating systems or combinations of operating systems.

Computer 802 can optionally comprise emulation technologies. For example, a hypervisor (not shown) or other intermediary can emulate a hardware environment for operating system 830, and the emulated hardware can optionally be different from the hardware illustrated in FIG. 8. In such an embodiment, operating system 830 can comprise one virtual machine (VM) of multiple VMs hosted at computer 802. Furthermore, operating system 830 can provide runtime environments, such as the Java runtime environment or the .NET framework, for applications 832. Runtime environments are consistent execution environments that allow applications 832 to run on any operating system that includes the runtime environment. Similarly, operating system 830 can support containers, and applications 832 can be in the form of containers, which are lightweight, standalone, executable packages of software that include, e.g., code, runtime, system tools, system libraries and settings for an application.

Further, computer 802 can be enabled with a security module, such as a trusted processing module (TPM). For instance, with a TPM, boot components hash next in time boot components, and wait for a match of results to secured values, before loading a next boot component. This process can take place at any layer in the code execution stack of computer 802, e.g., applied at the application execution level or at the operating system (OS) kernel level, thereby enabling security at any level of code execution.

A user can enter commands and information into the computer 802 through one or more wired/wireless input devices, e.g., a keyboard 838, a touch screen 840, and a pointing device, such as a mouse 842. Other input devices (not shown) can include a microphone, an infrared (IR) remote control, a radio frequency (RF) remote control, or other remote control, a joystick, a virtual reality controller and/or virtual reality headset, a game pad, a stylus pen, an image input device, e.g., camera(s), a gesture sensor input device, a vision movement sensor input device, an emotion or facial detection device, a biometric input device, e.g., fingerprint or iris scanner, or the like. These and other input devices are often connected to the processing unit 804 through an input device interface 844 that can be coupled to the system bus 808, but can be connected by other interfaces, such as a parallel port, an IEEE 1394 serial port, a game port, a USB port, an IR interface, a BLUETOOTH® interface, etc.

A monitor 846 or other type of display device can be also connected to the system bus 808 via an interface, such as a video adapter 848. In addition to the monitor 846, a computer typically includes other peripheral output devices (not shown), such as speakers, printers, etc.

The computer 802 can operate in a networked environment using logical connections via wired and/or wireless communications to one or more remote computers, such as a remote computer(s) 850. The remote computer(s) 850 can be a workstation, a server computer, a router, a personal computer, portable computer, microprocessor-based entertainment appliance, a peer device or other common network node, and typically includes many or all of the elements described relative to the computer 802, although, for purposes of brevity, only a memory/storage device 852 is illustrated. The logical connections depicted include wired/wireless connectivity to a local area network (LAN) 854 and/or larger networks, e.g., a wide area network (WAN) 856. Such LAN and WAN networking environments are commonplace in offices and companies, and facilitate enterprise-wide computer networks, such as intranets, all of which can connect to a global communications network, e.g., the Internet.

When used in a LAN networking environment, the computer 802 can be connected to the local network 854 through a wired and/or wireless communication network interface or adapter 858. The adapter 858 can facilitate wired or wireless communication to the LAN 854, which can also include a wireless access point (AP) disposed thereon for communicating with the adapter 858 in a wireless mode.

When used in a WAN networking environment, the computer 802 can include a modem 860 or can be connected to a communications server on the WAN 856 via other means for establishing communications over the WAN 856, such as by way of the Internet. The modem 860, which can be internal or external and a wired or wireless device, can be connected to the system bus 808 via the input device interface 844. In a networked environment, program modules depicted relative to the computer 802 or portions thereof, can be stored in the remote memory/storage device 852. It will be appreciated that the network connections shown are example and other means of establishing a communications link between the computers can be used.

When used in either a LAN or WAN networking environment, the computer 802 can access cloud storage systems or other network-based storage systems in addition to, or in place of, external storage devices 816 as described above. Generally, a connection between the computer 802 and a cloud storage system can be established over a LAN 854 or WAN 856 e.g., by the adapter 858 or modem 860, respectively. Upon connecting the computer 802 to an associated cloud storage system, the external storage interface 826 can, with the aid of the adapter 858 and/or modem 860, manage storage provided by the cloud storage system as it would other types of external storage. For instance, the external storage interface 826 can be configured to provide access to cloud storage sources as if those sources were physically connected to the computer 802.

The computer 802 can be operable to communicate with any wireless devices or entities operatively disposed in wireless communication, e.g., a printer, scanner, desktop and/or portable computer, portable data assistant, communications satellite, any piece of equipment or location associated with a wirelessly detectable tag (e.g., a kiosk, news stand, store shelf, etc.), and telephone. This can include Wireless Fidelity (Wi-Fi) and BLUETOOTH® wireless technologies. Thus, the communication can be a predefined structure as with a conventional network or simply an ad hoc communication between at least two devices.

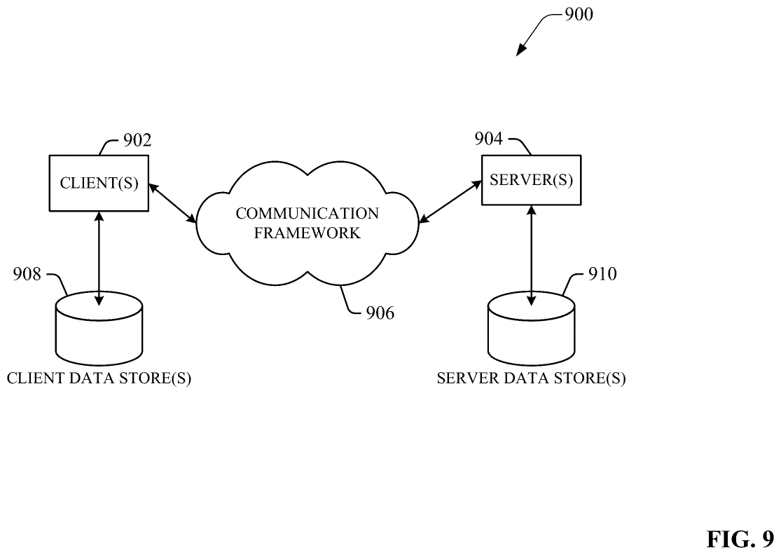

Referring now to FIG. 9, there is illustrated a schematic block diagram of a computing environment 900 in accordance with this specification. The system 900 includes one or more client(s) 902, (e.g., computers, smart phones, tablets, cameras, PDA's). The client(s) 902 can be hardware and/or software (e.g., threads, processes, computing devices). The client(s) 902 can house cookie(s) and/or associated contextual information by employing the specification, for example.

The system 900 also includes one or more server(s) 904. The server(s) 904 can also be hardware or hardware in combination with software (e.g., threads, processes, computing devices). The servers 904 can house threads to perform transformations of media items by employing aspects of this disclosure, for example. One possible communication between a client 902 and a server 904 can be in the form of a data packet adapted to be transmitted between two or more computer processes wherein data packets may include coded analyzed headspaces and/or input. The data packet can include a cookie and/or associated contextual information, for example. The system 900 includes a communication framework 906 (e.g., a global communication network such as the Internet) that can be employed to facilitate communications between the client(s) 902 and the server(s) 904.

Communications can be facilitated via a wired (including optical fiber) and/or wireless technology. The client(s) 902 are operatively connected to one or more client data store(s) 908 that can be employed to store information local to the client(s) 902 (e.g., cookie(s) and/or associated contextual information). Similarly, the server(s) 904 are operatively connected to one or more server data store(s) 910 that can be employed to store information local to the servers 904. Further, the client(s) 902 can be operatively connected to one or more server data store(s) 910.

In one exemplary implementation, a client 902 can transfer an encoded file, (e.g., encoded media item), to server 904. Server 904 can store the file, decode the file, or transmit the file to another client 902. It is noted that a client 902 can also transfer uncompressed file to a server 904 and server 904 can compress the file and/or transform the file in accordance with this disclosure. Likewise, server 904 can encode information and transmit the information via communication framework 906 to one or more clients 902.

The illustrated aspects of the disclosure can also be practiced in distributed computing environments where certain tasks are performed by remote processing devices that are linked through a communications network. In a distributed computing environment, program modules can be located in both local and remote memory storage devices.

The above description includes non-limiting examples of the various embodiments. It is, of course, not possible to describe every conceivable combination of components or methods for purposes of describing the disclosed subject matter, and one skilled in the art can recognize that further combinations and permutations of the various embodiments are possible. The disclosed subject matter is intended to embrace all such alterations, modifications, and variations that fall within the spirit and scope of the appended claims.

With regard to the various functions performed by the above-described components, devices, circuits, systems, etc., the terms (including a reference to a “means”) used to describe such components are intended to also include, unless otherwise indicated, any structure(s) which performs the specified function of the described component (e.g., a functional equivalent), even if not structurally equivalent to the disclosed structure. In addition, while a particular feature of the disclosed subject matter may have been disclosed with respect to only one of several implementations, such feature can be combined with one or more other features of the other implementations as may be desired and advantageous for any given or particular application.

The terms “exemplary” and/or “demonstrative” as used herein are intended to mean serving as an example, instance, or illustration. For the avoidance of doubt, the subject matter disclosed herein is not limited by such examples. In addition, any aspect or design described herein as “exemplary” and/or “demonstrative” is not necessarily to be construed as preferred or advantageous over other aspects or designs, nor is it meant to preclude equivalent structures and techniques known to one skilled in the art. Furthermore, to the extent that the terms “includes,” “has,” “contains,” and other similar words are used in either the detailed description or the claims, such terms are intended to be inclusive—in a manner similar to the term “comprising” as an open transition word—without precluding any additional or other elements.

The term “or” as used herein is intended to mean an inclusive “or” rather than an exclusive “or.” For example, the phrase “A or B” is intended to include instances of A, B, and both A and B. Additionally, the articles “a” and “an” as used in this application and the appended claims should generally be construed to mean “one or more” unless either otherwise specified or clear from the context to be directed to a singular form.

The term “set” as employed herein excludes the empty set, i.e., the set with no elements therein. Thus, a “set” in the subject disclosure includes one or more elements or entities. Likewise, the term “group” as utilized herein refers to a collection of one or more entities.

The description of illustrated embodiments of the subject disclosure as provided herein, including what is described in the Abstract, is not intended to be exhaustive or to limit the disclosed embodiments to the precise forms disclosed. While specific embodiments and examples are described herein for illustrative purposes, various modifications are possible that are considered within the scope of such embodiments and examples, as one skilled in the art can recognize. In this regard, while the subject matter has been described herein in connection with various embodiments and corresponding drawings, where applicable, it is to be understood that other similar embodiments can be used or modifications and additions can be made to the described embodiments for performing the same, similar, alternative, or substitute function of the disclosed subject matter without deviating therefrom. Therefore, the disclosed subject matter should not be limited to any single embodiment described herein, but rather should be construed in breadth and scope in accordance with the appended claims below.

Further aspects of the invention are provided by the subject matter of the following clauses:
1. A vehicle, comprising:
a memory that stores computer executable components; and
a processor that executes the computer executable components stored in the memory, wherein the computer executable components comprise:
a health component that, using a health sensor, determines one or more health metrics associated with an occupant of the vehicle;
an activation component that, based on a defined activation criterion associated with the one or more health metrics being determined to be satisfied, activates an autonomous driving mode;
a navigation component that, in response to activation of the autonomous driving mode, determines a route from a current location of the vehicle to a defined location, wherein the defined location is determined based on the one or more health metrics; and
an autonomous driving component that, using the route, autonomously navigates the vehicle to the defined location.
2. The vehicle of any preceding clause, wherein the health sensor comprises a sensor of a steering wheel of the vehicle.
3. The vehicle of any preceding clause, wherein the health sensor comprises an electrocardiogram sensor.
4. The vehicle of any preceding clause, wherein the health sensor comprises a pulse oximeter sensor.
5. The vehicle of any preceding clause, wherein the health sensor comprises a camera in an interior of the vehicle.
6. The vehicle of any preceding clause, wherein the health sensor comprises a wearable device, worn by the occupant of the vehicle, and communicatively coupled to the vehicle.
7. The vehicle of any preceding clause, wherein the health sensor comprises a glucose monitoring sensor.
8. The vehicle of any preceding clause, wherein the defined activation criterion comprises threshold likely incapacitation of the occupant of the vehicle.
9. The vehicle of any preceding clause, wherein the route is determined to comprise a shortest distance from the current location the defined location.
10. The vehicle of any preceding clause, wherein the route is determined to comprise a shortest amount of travel time between the current location and the defined location.
11. The vehicle of any preceding clause, wherein the route is determined based on a predicted treatment time at a group of defined locations, comprising the defined location.
12. The vehicle of any preceding clause, wherein the computer executable components further comprise:
a communication component that transmits, to other vehicles on the route, other than the vehicle, an instruction to clear a path for the vehicle along the route.
13. The vehicle of any preceding clause, wherein the computer executable components further comprise:
a communication component that transmits an alert, associated with the activation of the autonomous driving mode or the one or more health metrics, to the defined location.
14. The vehicle of any preceding clause, wherein the computer executable components further comprise:
a communication component that transmits an alert, associated with the activation of the autonomous driving mode or the one or more health metrics, to a registered recipient associated with the occupant of the vehicle.
15. The vehicle of any preceding clause, wherein the alert comprises the current location and the defined location.
16. The vehicle of clause 1 above with any set of combinations of the vehicles 2-15 above.
17. A method, comprising:
using a health sensor, determining, by a vehicle comprising a processor, one or more health metrics associated with an occupant of the vehicle;
based on a defined activation criterion associated with the one or more health metrics being determined to be satisfied, activating, by the vehicle, an autonomous driving mode;
in response to activation of the autonomous driving mode, determining, by the vehicle, a route from a current location of the vehicle to a defined location, wherein the defined location is determined based on the one or more health metrics; and
using the route, autonomously navigating, by the vehicle, the vehicle to the defined location.
18. The method of any preceding clause, wherein the health sensor comprises a sensor of a steering wheel of the vehicle.
19. The method of any preceding clause, wherein the health sensor comprises a wearable device, worn by the occupant of the vehicle, and communicatively coupled to the vehicle.
20. The method of clause 17 above with any set of combinations of the methods of clauses 18-19 above.
21. A non-transitory machine-readable medium, comprising executable instructions that, when executed by a processor, facilitate performance of operations, comprising:
using a health sensor, determining one or more health metrics associated with an occupant of a vehicle;
based on a defined activation criterion associated with the one or more health metrics being determined to be satisfied, activating an autonomous driving mode of the vehicle;
in response to activation of the autonomous driving mode, determining a route from a current location of the vehicle to a defined location, wherein the defined location is determined based on the one or more health metrics; and
using the route, autonomously navigating the vehicle to the defined location.
22. The non-transitory machine-readable medium of any preceding clause, wherein the defined activation criterion comprises threshold likely incapacitation of the occupant of the vehicle.

What is claimed is:

1. A vehicle, comprising:
a memory that stores computer executable components; and
a processor that executes the computer executable components stored in the memory, wherein the computer executable components comprise:
a health component that, using a health sensor, determines one or more health metrics associated with an occupant of the vehicle;
an activation component that, based on a defined activation criterion associated with the one or more health metrics being determined to be satisfied, activates an autonomous driving mode;
a navigation component that, in response to activation of the autonomous driving mode, determines a route from a current location of the vehicle to a defined location, wherein the defined location is determined based on the one or more health metrics; and
an autonomous driving component that, using the route, autonomously navigates the vehicle to the defined location.

2. The vehicle of claim 1, wherein the health sensor comprises a sensor of a steering wheel of the vehicle.

3. The vehicle of claim 1, wherein the health sensor comprises an electrocardiogram sensor.

4. The vehicle of claim 1, wherein the health sensor comprises a pulse oximeter sensor.

5. The vehicle of claim 1, wherein the health sensor comprises a camera in an interior of the vehicle.

6. The vehicle of claim 1, wherein the health sensor comprises a wearable device, worn by the occupant of the vehicle, and communicatively coupled to the vehicle.

7. The vehicle of claim 1, wherein the health sensor comprises a glucose monitoring sensor.

8. The vehicle of claim 1, wherein the defined activation criterion comprises threshold likely incapacitation of the occupant of the vehicle.

9. The vehicle of claim 1, wherein the route is determined to comprise a shortest distance from the current location the defined location.

10. The vehicle of claim 1, wherein the route is determined to comprise a shortest amount of travel time between the current location and the defined location.

11. The vehicle of claim 1, wherein the route is determined based on a predicted treatment time at a group of defined locations, comprising the defined location.

12. The vehicle of claim 1, wherein the computer executable components further comprise:
a communication component that transmits, to other vehicles on the route, other than the vehicle, an instruction to clear a path for the vehicle along the route.

13. The vehicle of claim 1, wherein the computer executable components further comprise:
a communication component that transmits an alert, associated with the activation of the autonomous driving mode or the one or more health metrics, to the defined location.

14. The vehicle of claim 1, wherein the computer executable components further comprise:
a communication component that transmits an alert, associated with the activation of the autonomous driving mode or the one or more health metrics, to a registered recipient associated with the occupant of the vehicle.

15. The vehicle of claim 14, wherein the alert comprises the current location and the defined location.

16. A method, comprising:
using a health sensor, determining, by a vehicle comprising a processor, one or more health metrics associated with an occupant of the vehicle;
based on a defined activation criterion associated with the one or more health metrics being determined to be satisfied, activating, by the vehicle, an autonomous driving mode;
in response to activation of the autonomous driving mode, determining, by the vehicle, a route from a current location of the vehicle to a defined location, wherein the defined location is determined based on the one or more health metrics; and
using the route, autonomously navigating, by the vehicle, the vehicle to the defined location.

17. The method of claim 16, wherein the health sensor comprises a sensor of a steering wheel of the vehicle.

18. The method of claim 16, wherein the health sensor comprises a wearable device, worn by the occupant of the vehicle, and communicatively coupled to the vehicle.

19. A non-transitory machine-readable medium, comprising executable instructions that, when executed by a processor, facilitate performance of operations, comprising:
using a health sensor, determining one or more health metrics associated with an occupant of a vehicle;
based on a defined activation criterion associated with the one or more health metrics being determined to be satisfied, activating an autonomous driving mode of the vehicle;
in response to activation of the autonomous driving mode, determining a route from a current location of the vehicle to a defined location, wherein the defined location is determined based on the one or more health metrics; and
using the route, autonomously navigating the vehicle to the defined location.

20. The non-transitory machine-readable medium of claim 19, wherein the defined activation criterion comprises threshold likely incapacitation of the occupant of the vehicle.
